(19) United States
(12) Patent Application Publication
(10) Pub. No.: US 2005/0106620 A1
(43) Pub. Date: May 19, 2005

(54) Novel method of diagnosing, monitoring, staging, imaging and treating cancer

(76) Inventors: Susana Salceda, San Jose, CA (US); Robert Cafferkey, San Jose, CA (US); Xing Jian Lou, Mountain View, CA (US); Herve Recipon, San Francisco, CA (US); Yongming Sun, San Jose, CA (US)

Correspondence Address: LICATA & TYRRELL P.C., 66 E. MAIN STREET, MARLTON, NJ, 08053, US

(21) Appl. No.: 11/022,712

(22) Filed: Dec. 27, 2004

Related U.S. Application Data
(63) Continuation of application No. 09/717,883, filed on Nov. 21, 2000.
(60) Provisional application No. 60/166,818, filed on Nov. 22, 1999.

(51) Int. Cl. C12Q 1/68; G01N 33/574

(52) U.S. Cl. 435/6; 435/7.23

(57) ABSTRACT

The present invention provides new markers and methods for detecting, diagnosing, monitoring, staging, prognosticating, imaging and treating cancer.

### INTRODUCTION

This application claims the benefit of priority from U.S. provisional application Ser. No. 60/166,818, filed Nov. 22, 1999.

### FIELD OF THE INVENTION

This invention relates, in part, to newly developed assays for detecting, diagnosing, monitoring, staging, prognosticating, imaging and treating cancers, particularly ovarian cancer.

### BACKGROUND OF THE INVENTION

In women, gynecologic cancers account for more than one-fourth of the malignancies. Carcinoma of the ovary is a very common gynecologic cancer. Approximately one in 70 women will develop ovarian cancer during her lifetime and in 1995 there were an estimated 14,500 deaths from ovarian cancer. In fact, ovarian cancer causes more deaths than any other cancer of the female reproductive system.

Ovarian cancer often does not cause any noticeable symptoms. Some possible warning signals include an enlarged abdomen due to an accumulation of fluid or vague digestive disturbances (discomfort, gas or distention) in women over 40; rarely there will be abnormal vaginal bleeding.

Periodic, complete pelvic examinations are important in the detection of ovarian cancer; a Pap test does not detect ovarian cancer. Annual pelvic exams are recommended for women over 40.

Procedures used for detecting, diagnosing, monitoring, staging, and prognosticating cancers are of critical importance to the outcome of the patient. Patients diagnosed early generally have a much greater five-year survival rate as compared to the survival rate for patients diagnosed with distant metastasized cancer. New diagnostic methods which are more sensitive and specific for detecting early cancers are clearly needed.

Cancer patients are closely monitored following initial therapy and during adjuvant therapy to determine response to therapy and to detect persistent or recurrent disease or metastasis. Thus, there is also clearly a need for cancer markers which are more sensitive and specific in detecting cancer recurrence.

Another important step in managing cancer is to determine the stage of the patient's disease. Stage determination has potential prognostic value and provides criteria for designing optimal therapy. Generally, pathological staging of cancer is preferable over clinical staging because the former gives a more accurate prognosis. However, clinical staging would be preferred were it at least as accurate as pathological staging because it does not depend on an invasive procedure to obtain tissue for pathological evaluation. Staging of cancer would be improved by detecting new markers in cells, tissues or bodily fluids which could differentiate between different stages of invasion.

A new marker, referred to herein as Ovr107, has now been identified for use in diagnosing, monitoring, staging, imaging and treating various cancers, and in particular ovarian cancer. Accordingly, the present invention relates to new methods for detecting, diagnosing, monitoring, staging, prognosticating, in vivo imaging and treating cancer via Ovr107. Ovr107 refers, among other things, to native proteins expressed by the gene comprising the polynucleotide sequence of SEQ ID NO:1. By “Ovr107” it is also meant herein polynucleotides which, due to degeneracy in genetic coding, comprise variations in nucleotide sequence as compared to SEQ ID NO: 1, but which still encode the same protein. In the alternative, what is meant by Ovr107 as used herein, means the native mRNA encoded by the gene comprising SEQ ID NO:1 or it can refer to the actual gene comprising SEQ ID NO:1, or levels of a polynucleotide which is capable of hybridizing under stringent conditions to the antisense sequence of SEQ ID NO:1.

Other objects, features, advantages and aspects of the present invention will become apparent to those of skill in the art from the following description. It should be understood, however, that the following description and the specific examples, while indicating preferred embodiments of the invention, are given by way of illustration only. Various changes and modifications within the spirit and scope of the disclosed invention will become readily apparent to those skilled in the art from reading the following description and from reading the other parts of the present disclosure.

### SUMMARY OF THE INVENTION

Toward these ends, and others, it is an object of the present invention to provide a diagnostic marker for cancer comprising Ovr107.

Further provided is a method for diagnosing the presence cancer by analyzing for changes in levels of Ovr107 in cells, tissues or bodily fluids compared with levels of Ovr107 in preferably the same cells, tissues, or bodily fluid type of a normal human control, wherein a change in levels of Ovr107 in the patient versus the normal human control is associated with cancer.

Further provided is a method of diagnosing metastatic cancer in a patient having cancer which is not known to have metastasized by identifying a human patient suspected of having cancer that has metastasized; analyzing a sample of cells, tissues, or bodily fluid from such patient for Ovr107; comparing the Ovr107 levels in such cells, tissues, or bodily fluid with levels of Ovr107 in preferably the same cells, tissues, or bodily fluid type of a normal human control, wherein an increase in Ovr107 levels in the patient versus the normal human control is associated with cancer which has metastasized.

Also provided by the invention is a method of staging cancer in a human with cancer by identifying a human patient having cancer; analyzing a sample of cells, tissues, or bodily fluid from such patient for Ovr107; comparing Ovr107 levels in such cells, tissues, or bodily fluid with levels of Ovr107 in preferably the same cells, tissues, or bodily fluid type of a normal human control, wherein an increase in Ovr107 levels in the patient versus the normal human control is associated with a cancer which is progressing and a decrease in the levels of Ovr107 is associated with a cancer which is regressing or in remission.

Further provided is a method of monitoring cancer in a human patient for the onset of metastasis. The method comprises identifying a human patient having cancer that is not known to have metastasized; periodically analyzing cells, tissues, or bodily fluid from such patient for Ovr107; comparing the Ovr107 levels in such cells, tissue, or bodily fluid with levels of Ovr107 in preferably the same cells, tissues, or bodily fluid type of a normal human control, wherein an increase in Ovr107 levels in the patient versus the normal human control is associated with a cancer which has metastasized.

Further provided is a method of monitoring the change in stage of cancer in a human patient by looking at levels of Ovr107 in the human patient. The method comprises identifying a human patient having cancer; periodically analyzing cells, tissues, or bodily fluid from such patient for Ovr107; comparing the Ovr107 levels in such cells, tissue, or bodily fluid with levels of Ovr107 in preferably the same cells, tissues, or bodily fluid type of a normal human control, wherein an increase in Ovr107 levels in the patient versus the normal human control is associated with a cancer which is progressing and a decrease in the levels of Ovr107 is associated with a cancer which is regressing or in remission.

Further provided are methods of designing new therapeutic agents targeted to Ovr107 for use in imaging and treating cancer. For example, in one embodiment, therapeutic agents such as antibodies targeted against Ovr107 or fragments of such antibodies can be used to treat, detect or image localization of Ovr107 in a patient for the purpose of detecting or diagnosing a disease or condition. In this embodiment, an increase in the amount of labeled antibody detected as compared to normal tissue would be indicative of tumor metastases or growth. Such antibodies can be polyclonal, monoclonal, or omniclonal or prepared by molecular biology techniques. The term “antibody”, as used herein and throughout the instant specification is also meant to include aptamers and single-stranded oligonucleotides such as those derived from an in vitro evolution protocol referred to as SELEX and well known to those skilled in the art. Antibodies can be labeled with a variety of detectable labels including, but not limited to, radioisotopes and paramagnetic metals. Therapeutics agents such as small molecules and antibodies or fragments thereof which decrease the concentration and/or activity of Ovr107 can also be used in the treatment of diseases characterized by overexpression of Ovr107. Such agents can be readily identified in accordance with teachings herein.

Other objects, features, advantages and aspects of the present invention will become apparent to those of skill in the art from the following description. It should be understood, however, that the following description and the specific examples, while indicating preferred embodiments of the invention, are given by way of illustration only. Various changes and modifications within the spirit and scope of the disclosed invention will become readily apparent to those skilled in the art from reading the following description and from reading the other parts of the present disclosure.

### DESCRIPTION OF THE INVENTION

The present invention relates to, inter alia, diagnostic assays and methods, both quantitative and qualitative for detecting, diagnosing, monitoring, staging, prognosticating, in vivo imaging and treating cancers by comparing levels of Ovr107 with those of Ovr107 in a normal human control. Ovr107 refers, among other things, to native proteins expressed by the gene comprising the polynucleotide sequence of SEQ ID NO:1. By “Ovr107” it is also meant herein polynucleotides which, due to degeneracy in genetic coding, comprise variations in nucleotide sequence as compared to SEQ ID NO: 1, but which still encode the same protein. In the alternative, what is meant by Ovr107 as used herein, means the native mRNA encoded by the gene comprising SEQ ID NO:1 or it can refer to the actual gene comprising SEQ ID NO:1, or levels of a polynucleotide which is capable of hybridizing under stringent conditions to the antisense sequence of SEQ ID NO:1. Such levels are preferably measured in at least one of, cells, tissues and/or bodily fluids, including determination of normal and abnormal levels. Thus, for instance, a diagnostic assay in accordance with the invention for diagnosing over-expression of Ovr107 protein compared to normal control bodily fluids, cells, or tissue samples can be used to diagnose the presence of cancers, including ovarian cancer. Ovr107 may be measured alone in the methods of the invention, or, more preferably, in combination with other diagnostic markers for cancer. Thus, it is preferred that the methods of the present invention be employed in combination with measurement of the levels of other cancer markers as well as Ovr107. Other cancer markers, in addition to Ovr107, useful in the present invention will depend on the cancer being tested and are known to those of skill in the art.

Detection of Ovr107 is particularly useful in ovarian cancer. However, this marker is also useful in the diagnosis, prognosis, staging, imaging and treatment of other types of cancer.

Diagnostic Assays

The present invention provides methods for diagnosing the presence of cancer, including ovarian cancer, by analyzing for changes in levels of Ovr107 in cells, tissues or bodily fluids from a human patient compared with levels of Ovr107 in cells, tissues or bodily fluids of preferably the same type from a normal human control, wherein an increase in levels of Ovr107 in the patient versus the normal human control is associated with the presence of cancer.

Without limiting the instant invention, typically, for a quantitative diagnostic assay a positive result indicating the patient being tested has cancer is one in which cells, tissues, or bodily fluid levels of a cancer marker, such as Ovr107, are at least two times higher, and most preferably are at least five times higher, than in preferably the same cells, tissues, or bodily fluid of a normal human control.

The present invention also provides a method of diagnosing metastatic cancer, including metastatic ovarian cancer, in a patient having a cancer which has not yet metastasized. In the method of the present invention, a human cancer patient suspected of having cancer which may have metastasized (but which was not previously known to have metastasized) is identified. This is accomplished by a variety of means known to those of skill in the art. For example, in the case of ovarian cancer, patients are typically diagnosed with ovarian cancer following traditional detection methods.

In the present invention, determining the presence of Ovr107 in cells, tissues, or bodily fluid, is particularly useful for discriminating between cancers which have not metastasized and cancers which have metastasized. Existing techniques have difficulty discriminating between a cancer which has metastasized and a cancer which has not metastasized. However, proper treatment selection is often dependent upon such knowledge.

In the present invention, one of the cancer marker levels measured in cells, tissues, or bodily fluid of a human patient is Ovr107. Levels in the human patient are compared with levels of Ovr107 in preferably the same cells, tissue, or bodily fluid type of a normal human control. That is, if the cancer marker being observed is Ovr107 in serum, this level is preferably compared with the level of Ovr107 in serum of a normal human control. An increase in Ovr107 in the human patient versus the normal human control is associated with a cancer which has metastasized.

Without limiting the instant invention, typically, for a quantitative diagnostic assay a positive result indicating the cancer in the patient being tested or monitored has metastasized is one in which cells, tissues, or bodily fluid levels of a cancer marker, such as Ovr107, are at least two times higher, and more preferably are at least five times higher, than in preferably the same cells, tissues, or bodily fluid of a normal human control.

Normal human control as used herein includes a human patient without cancer and/or non cancerous samples from the patient; in the methods for diagnosing metastasis or monitoring for metastasis, normal human control preferably includes samples from a human patient that is determined by reliable methods to have a cancer such as ovarian cancer which has not metastasized, such as samples from the same patient prior to metastasis.

Staging

The invention also provides a method of staging cancers in a human patient.

The method comprises identifying a human patient having cancer and analyzing a sample of cells, tissues, or bodily fluid from such patient for Ovr107. The measured Ovr107 levels are then compared to levels of Ovr107 in preferably the same cells, tissues, or bodily fluid type of a normal human control, wherein an increase in Ovr107 levels in the human patient versus the normal human control is associated with a cancer which is progressing and a decrease in the levels of Ovr107 is associated with a cancer which is regressing or in remission.

Monitoring

Further provided is a method of monitoring cancer in a human patient for the onset of metastasis. The method comprises identifying a human patient having cancer that is not known to have metastasized; periodically analyzing cells, tissues, or bodily fluid from such patient for Ovr107; and comparing the Ovr107 levels in such cells, tissue, or bodily fluid with levels of Ovr107 in preferably the same cells, tissues, or bodily fluid type of a normal human control, wherein an increase in Ovr107 levels in the patient versus the normal human control is associated with a cancer which has metastasized.

Further provided by this invention is a method of monitoring the change in stage of a cancer. The method comprises identifying a human patient having cancer; periodically analyzing cells, tissues, or bodily fluid from such patient for Ovr107; and comparing the Ovr107 levels in such cells, tissue, or bodily fluid with levels of Ovr107 in preferably the same cells, tissues, or bodily fluid type of a normal human control, wherein an increase in Ovr107 levels in the patient versus the normal human control is associated with a cancer which is progressing in stage and a decrease in the levels of Ovr107 is associated with a cancer which is regressing in stage or in remission.

Monitoring such patients for onset of metastasis is periodic and preferably done on a quarterly basis. However, this may be performed more or less frequently depending on the cancer, the particular patient, and the stage of the cancer.

Prognostic Testing and Clinical Trial Monitoring

The methods described herein can further be utilized as prognostic assays to identify subjects having or at risk of developing a disease or disorder associated with increased levels of Ovr107. The present invention provides a method in which a test sample is obtained from a human patient and Ovr107 is detected. The presence of higher Ovr107 levels as compared to normal human controls is diagnostic for the human patient being at risk for developing cancer, particularly ovarian cancer.

The effectiveness of therapeutic agents to decrease expression or activity of Ovr107 can also be monitored by analyzing levels of expression of Ovr107 in a human patient in clinical trials or in in vitro screening assays such as in human cells. In this way, the gene expression pattern can serve as a marker, indicative of the physiological response of the human patient, or cells as the case may be, to the agent being tested.

Detection of Genetic Lesions or Mutations

The methods of the present invention can also be used to detect genetic lesions or mutations in Ovr107, thereby determining if a human with the genetic lesion is at risk for cancer or has cancer, particularly ovarian cancer. Genetic lesions can be detected, for example, by ascertaining the existence of a deletion and/or addition and/or substitution of one or more nucleotides from Ovr107, a chromosomal rearrangement of Ovr107, aberrant modification of Ovr107 (such as of the methylation pattern of the genomic DNA), the presence of a non-wild type splicing pattern of a mRNA transcript of Ovr107, allelic loss of Ovr107, and/or inappropriate post-translational modification of Ovr107 protein. Methods to detect such lesions in Ovr107 of this invention are known to those of skill in the art.

Assay Techniques

Assay techniques that can be used to determine levels of gene expression (including protein levels), such as Ovr107 of the present invention, in a sample derived from a human are well-known to those of skill in the art. Such assay methods include radioimmunoassays, reverse transcriptase PCR (RT-PCR) assays, immunohistochemistry assays, in situ hybridization assays, competitive-binding assays, Western Blot analyses, ELISA assays and proteomic approaches, 2-dimensional gel electrophoresis (2D electrophoresis) and non-gel based approaches such as mass spectrometry or protein interaction profiling. Among these, ELISAs are frequently preferred to diagnose a gene's expressed protein in biological fluids.

An ELISA assay initially comprises preparing an antibody, if not readily available from a commercial source, specific to Ovr107, preferably a monoclonal antibody. In addition a reporter antibody generally is prepared which binds specifically to Ovr107. The reporter antibody is attached to a detectable reagent such as a radioactive, fluorescent or enzymatic reagent, for example horseradish peroxidase enzyme or alkaline phosphatase.

To carry out the ELISA, antibody specific to Ovr107 is incubated on a solid support, e.g., a polystyrene dish, that binds the antibody. Any free protein binding sites on the dish are then covered by incubating with a non-specific protein such as bovine serum albumin. Next, the sample to be analyzed is incubated in the dish, during which time Ovr107 binds to the specific antibody attached to the polystyrene dish. Unbound sample is washed out with buffer. A reporter antibody specifically directed to Ovr107 and linked to a detectable reagent such as horseradish peroxidase is placed in the dish resulting in binding of the reporter antibody to any monoclonal antibody bound to Ovr107. Unattached reporter antibody is then washed out. Reagents for peroxidase activity, including a calorimetric substrate are then added to the dish. Immobilized peroxidase, linked to Ovr107 antibodies, produces a colored reaction product. The amount of color developed in a given time period is proportional to the amount of Ovr107 protein present in the sample. Quantitative results typically are obtained by reference to a standard curve.

A competition assay can also be employed wherein antibodies specific to Ovr107 are attached to a solid support and labeled Ovr107 and a sample derived from the patient or human control are passed over the solid support. The amount of label detected which is attached to the solid support can be correlated to a quantity of Ovr107 in the sample.

Using all or a portion of a nucleic acid sequence of Ovr107 of the present invention as a hybridization probe, nucleic acid methods can also be used to detect Ovr107 mRNA as a marker for cancer, including ovarian cancer. Polymerase chain reaction (PCR) and other nucleic acid methods, such as ligase chain reaction (LCR) and nucleic acid sequence based amplification (NASABA), can be used to detect malignant cells for diagnosis and monitoring of various malignancies. For example, reverse-transcriptase PCR (RT-PCR) is a powerful technique which can be used to detect the presence of a specific mRNA population in a complex mixture of thousands of other mRNA species. In RT-PCR, an mRNA species is first reverse transcribed to complementary DNA (cDNA) with use of the enzyme reverse transcriptase; the cDNA is then amplified as in a standard PCR reaction. RT-PCR can thus reveal by amplification the presence of a single species of mRNA. Accordingly, if the mRNA is highly specific for the cell that produces it, RT-PCR can be used to identify the presence of a specific type of cell.

Hybridization to clones or oligonucleotides arrayed on a solid support (i.e., gridding) can be used to both detect the expression of and quantitate the level of expression of that gene. In this approach, a cDNA encoding the Ovr107 gene is fixed to a substrate. The substrate may be of any suitable type including but not limited to glass, nitrocellulose, nylon or plastic. At least a portion of the DNA encoding the Ovr107 gene is attached to the substrate and then incubated with the analyte, which may be RNA or a complementary DNA (cDNA) copy of the RNA, isolated from the tissue of interest. Hybridization between the substrate bound DNA and the analyte can be detected and quantitated by several means including but not limited to radioactive labeling or fluorescence labeling of the analyte or a secondary molecule designed to detect the hybrid. Quantitation of the level of gene expression can be done by comparison of the intensity of the signal from the analyte compared with that determined from known standards. The standards can be obtained by in vitro transcription of the target gene, quantitating the yield, and then using that material to generate a standard curve.

Of the proteomic approaches, 2D electrophoresis is a technique well known to those in the art. Isolation of individual proteins from a sample such as serum is accomplished using sequential separation of proteins by different characteristics usually on polyacrylamide gels. First, proteins are separated by size using an electric current. The current acts uniformly on all proteins, so smaller proteins move farther on the gel than larger proteins. The second dimension applies a current perpendicular to the first and separates proteins not on the basis of size but on the specific electric charge carried by each protein. Since no two proteins with different sequences are identical on the basis of both size and charge, the result of a 2D separation is a square gel in which each protein occupies a unique spot. Analysis of the spots with chemical or antibody probes, or subsequent protein microsequencing can reveal the relative abundance of a given protein and the identity of the proteins in the sample.

The above tests can be carried out on samples derived from a variety cells, bodily fluids and/or tissue extracts (homogenates or solubilized tissue) obtained from the patient including tissue biopsy and autopsy material. Bodily fluids useful in the present invention include blood, urine, saliva, or any other bodily secretion or derivative thereof. Blood can include whole blood, plasma, serum, or any derivative of blood.

In Vivo Targeting of Ovr107/Cancer Therapy

Identification of Ovr107 is also useful in the rational design of new therapeutics for imaging and treating cancers, and in particular ovarian cancer. For example, in one embodiment, antibodies which specifically bind to Ovr107 can be raised and used in vivo in patients suspected of suffering from cancer. Antibodies which specifically bind a Ovr107 can be injected into a patient suspected of having cancer for diagnostic and/or therapeutic purposes. The preparation and use of antibodies for in vivo diagnosis is well known in the art. For example, antibody-chelators labeled with Indium-111 have been described for use in the radioimmunoscintographic imaging of carcinoembryonic antigen expressing tumors (Sumerdon et al. Nucl. Med. Biol. 1990 17:247-254). In particular, these antibody-chelators have been used in detecting tumors in patients suspected of having recurrent colorectal cancer (Griffin et al. J. Clin. One. 1991 9:631-640). Antibodies with paramagnetic ions as labels for use in magnetic resonance imaging have also been described (Lauffer, R. B. Magnetic Resonance in Medicine 1991 22:339-342). Antibodies directed against Ovr107 can be used in a similar manner. Labeled antibodies which specifically bind Ovr107 can be injected into patients suspected of having cancer for the purpose of diagnosing or staging of the disease status of the patient. The label used will be selected in accordance with the imaging modality to be used. For example, radioactive labels such as Indium-111, Technetium-99m or Iodine-131 can be used for planar scans or single photon emission computed tomography (SPECT). Positron emitting labels such as Fluorine-19 can be used in positron emission tomography. Paramagnetic ions such as Gadlinium (III) or Manganese (II) can be used in magnetic resonance imaging (MRI). Localization of the label permits determination of the spread of the cancer. The amount of label within an organ or tissue also allows determination of the presence or absence of cancer in that organ or tissue.

For patients diagnosed with cancer, and in particular ovarian cancer, injection of an antibody which specifically binds Ovr107 can also have a therapeutic benefit. The antibody may exert its therapeutic effect alone. Alternatively, the antibody can be conjugated to a cytotoxic agent such as a drug, toxin or radionuclide to enhance its therapeutic effect. Drug monoclonal antibodies have been described in the art for example by Garnett and Baldwin, Cancer Research 1986 46:2407-2412. The use of toxins conjugated to monoclonal antibodies for the therapy of various cancers has also been described by Pastan et al. Cell 1986 47:641-648. Yttrium-90 labeled monoclonal antibodies have been described for maximization of dose delivered to the tumor while limiting toxicity to normal tissues (Goodwin and Meares Cancer Supplement 1997 80:2675-2680). Other cytotoxic radionuclides including, but not limited to Copper-67, Iodine-131 and Rhenium-186 can also be used for labeling of antibodies against Ovr107.

Antibodies which can be used in these in vivo methods include polyclonal, monoclonal and omniclonal antibodies and antibodies prepared via molecular biology techniques. Antibody fragments and aptamers and single-stranded oligonucleotides such as those derived from an in vitro evolution protocol referred to as SELEX and well known to those skilled in the art can also be used.

Screening Assays

The present invention also provides methods for identifying modulators which bind to Ovr107 protein or have a modulatory effect on the expression or activity of Ovr107 protein. Modulators which decrease the expression or activity of Ovr107 protein are believed to be useful in treating cancer. Such screening assays are known to those of skill in the art and include, without limitation, cell-based assays and cell free assays.

Small molecules predicted via computer imaging to specifically bind to regions of Ovr107 can also be designed, synthesized and tested for use in the imaging and treatment of cancer. Further, libraries of molecules can be screened for potential anticancer agents by assessing the ability of the molecule to bind to Ovr107. Molecules identified in the library as being capable of binding to Ovr107 are key candidates for further evaluation for use in the treatment of cancer. In a preferred embodiment, these molecules will downregulate expression and/or activity of Ovr107 in cells.

Adoptive Immunotherapy and Vaccines

Adoptive immunotherapy of cancer refers to a therapeutic approach in which immune cells with an antitumor reactivity are administered to a tumor-bearing host, with the aim that the cells mediate either directly or indirectly, the regression of an established tumor. Transfusion of lymphocytes, particularly T lymphocytes, falls into this category and investigators at the National Cancer Institute (NCI) have used autologous reinfusion of peripheral blood lymphocytes or tumor-infiltrating lymphocytes (TIL), T cell cultures from biopsies of subcutaneous lymph nodules, to treat several human cancers (Rosenberg, S. A., U.S. Pat. No. 4,690,914, issued Sep. 1, 1987; Rosenberg, S. A., et al., 1988, N. England J. Med. 319:1676-1680).

The present invention relates to compositions and methods of adoptive immunotherapy for the prevention and/or treatment of cancer in humans using macrophages sensitized to the antigenic Ovr107 molecules, with or without non-covalent complexes of heat shock protein (hsp). Antigenicity or immunogenicity of Ovr107 is readily confirmed by the ability of the Ovr107 protein or a fragment thereof to raise antibodies or educate naive effector cells, which in turn lyse target cells expressing the antigen (or epitope).

Cancer cells are, by definition, abnormal and contain proteins which should be recognized by the immune system as foreign since they are not present in normal tissues. However, the immune system often seems to ignore this abnormality and fails to attack tumors. The foreign Ovr107 proteins that are produced by the cancer cells can be used to reveal their presence. Ovr107 is broken into short fragments, called tumor antigens, which are displayed on the surface of the cell. These tumor antigens are held or presented on the cell surface by molecules called MHC, of which there are two types: class I and II. Tumor antigens in association with MHC class I molecules are recognized by cytotoxic T cells while antigen-MHC class II complexes are recognized by a second subset of T cells called helper cells. These cells secrete cytokines which slow or stop tumor growth and help another type of white blood cell, B cells, to make antibodies against the tumor cells.

In adoptive immunotherapy, T cells or other antigen presenting cells (APCs) are stimulated outside the body (ex vivo), using the tumor specific Ovr107 antigen. The stimulated cells are then reinfused into the patient where they attack the cancerous cells. Research has shown that using both cytotoxic and helper T cells is far more effective than using either subset alone. Additionally, the Ovr107 antigen may be complexed with heat shock proteins to stimulate the APCs as described in U.S. Pat. No. 5,985,270.

The APCs can be selected from among those antigen presenting cells known in the art including, but not limited to, macrophages, dendritic cells, B lymphocytes, and a combination thereof, and are preferably macrophages. In a preferred use, wherein cells are autologous to the individual, autologous immune cells such as lymphocytes, macrophages or other APCs are used to circumvent the issue of whom to select as the donor of the immune cells for adoptive transfer. Another problem circumvented by use of autologous immune cells is graft versus host disease which can be fatal if unsuccessfully treated.

In adoptive immunotherapy with gene therapy, DNA of Ovr107 can be introduced into effector cells similarly as in conventional gene therapy. This can enhance the cytotoxicity of the effector cells to tumor cells as they have been manipulated to produce the antigenic protein resulting in improvement of the adoptive immunotherapy.

Ovr107 antigens of this invention are also useful as components of cancer vaccines. The vaccine comprises an immunogenically stimulatory amount of an Ovr107 antigen. Immunogenically stimulatory amount refers to that amount of antigen that is able to invoke the desired immune response in the recipient for the amelioration, or treatment of cancer. Effective amounts may be determined empirically by standard procedures well known to those skilled in the art.

The Ovr107 antigen may be provided in any one of a number of vaccine formulations which are designed to induce the desired type of immune response, e.g., antibody and/or cell mediated. Such formulations are known in the art and include, but are not limited to, formulations such as those described in U.S. Pat. No. 5,585,103. Vaccine formulations of the present invention used to stimulate immune responses can also include pharmaceutically acceptable adjuvants.

### EXAMPLE

The present invention is further described by the following example. The example is provided solely to illustrate the invention by reference to specific embodiments. This exemplification, while illustrating certain specific aspects of the invention, does not portray the limitations or circumscribe the scope of the disclosed invention.

Experiments described herein were carried out using standard techniques, which are well known and routine to those of skill in the art, except where otherwise described in detail. Routine molecular biology techniques were carried out as described in standard laboratory manuals, such as Sambrook et al., MOLECULAR CLONING: A LABORATORY MANUAL, 2nd Ed.; Cold Spring Harbor Laboratory Press, Cold Spring Harbor, N.Y. (1989).

Relative Quantitation of Gene Expression

Real-Time quantitative PCR with fluorescent Taqman probes is a quantitation detection system utilizing the 5′-3′ nuclease activity of Taq DNA polymerase. The method uses an internal fluorescent oligonucleotide probe (Taqman) labeled with a 5′ reporter dye and a downstream, 3′ quencher dye. During PCR, the 5′-3′ nuclease activity of Taq DNA polymerase releases the reporter, whose fluorescence can then be detected by the laser detector of the Model 7700 Sequence Detection System (PE Applied Biosystems, Foster City, Calif., USA).

Amplification of an endogenous control is used to standardize the amount of sample RNA added to the reaction and normalize for Reverse Transcriptase (RT) efficiency. Either cyclophilin, glyceraldehyde-3-phosphate dehydrogenase (GAPDH) or 18S ribosomal RNA (rRNA) is used as this endogenous control. To calculate relative quantitation between all the samples studied, the target RNA levels for one sample were used as the basis for comparative results (calibrator). Quantitation relative to the “calibrator” can be obtained using the standard curve method or the comparative method (User Bulletin #2: ABI PRISM 7700 Sequence Detection System).

The tissue distribution and the level of the target gene were evaluated for every sample in normal and cancer tissue. Total RNA was extracted from normal tissues, cancer tissues, and from cancers and the corresponding matched adjacent tissues. Subsequently, first strand cDNA was prepared with reverse transcriptase and the polymerase chain reaction was done using primers and Taqman probe specific to each target gene. The results are analyzed using the ABI PRISM 7700 Sequence Detector. The absolute numbers are relative levels of expression of the target gene in a particular tissue compared to the calibrator tissue.

Primers used for expression analysis include:

(SEQ ID NO:2)

| | |
|---|---|
| Reverse: | 5′-CCCAATAGCGGAAGTCGATCT-3′ |

(SEQ ID NO:3)

| | |
|---|---|
| Forward: | 5′-CACTCCCAGCCAGTCCAGAT-3′ |

(SEQ ID NO:4)

| | |
|---|---|
| Ovr107 Probe: | 5′-AATCTGCTCCGGCCCTGGTCTT-3′ |

The absolute numbers depicted in Table 1 are relative levels of expression of Ovr107 (also referred to as Clone ID 817834; Gene ID 403869) in 12 normal different tissues. All the values are compared to normal pancreas (calibrator). These RNA samples are commercially available pools, originated by pooling samples of a particular tissue from different individuals.

TABLE 1
Relative Levels of Ovr107 Expression in Pooled Samples

| TISSUE | NORMAL |
|---|---|
| Ascending Colon | 14.47 |
| Endometrium | 8.60 |
| Kidney | 2.64 |
| Liver | 0.08 |
| Ovary | 1.46 |
| Pancreas | 1.00 |
| Prostate | 17.22 |
| Small Intestine | 4.96 |
| Spleen | 2.86 |
| Stomach | 38.59 |
| Testis | 4.18 |
| Uterus | 13.45 |

The relative levels of expression in Table 1 show that Ovr107 is expressed in all the normal tissues analyzed. Stomach shows the highest relative expression level with 38.59, and liver the lowest expression value with 0.08.

The absolute numbers in Table 1 were obtained analyzing pools of samples of a particular tissue from different individuals. They can not be compared to the absolute numbers originated from RNA obtained from tissue samples of a single individual in Table 2.

The absolute numbers depicted in Table 2 are relative levels of expression of Ovr107 in 47 pairs of matching samples. All the values are compared to normal pancreas (calibrator). A matching pair is formed by mRNA from the cancer sample for a particular tissue and mRNA from the normal adjacent sample for that same tissue from the same individual. In addition, 12 unmatched cancer samples (from ovary) and 14 unmatched normal samples (from ovary) were also tested.

TABLE 2
Relative Levels of Ovr107 Expression in Individual Samples

| | | | Matching | |
|---|---|---|---|---|
| Sample | | | Normal | |
| ID | Tissue | Cancer | Adjacent | Normal |
| Ovr103X | Ovary 1 | 93.4 | | 1.30 |
| Ovr1028 | Ovary 2 | 182.90 | | |
| Ovr1005O | Ovary 3 | 113.31 | | |
| Ovr1040 | Ovary 4 | 132.50 | | |
| Ovr130X | Ovary 5 | 14.98 | | |
| Ovr638A | Ovary 6 | 102.89 | | |
| Ovr773O | Ovary 7 | 57.68 | | |
| OvrA1B | Ovary 8 | 50.91 | | |
| OvrA1C | Ovary 9 | 68.59 | | |
| Ovr1157 | Ovary 10 | 146.02 | | |
| Ovr1118 | Ovary 11 | 3.85 | | |
| OvrC360 | Ovary 12 | 2.91 | | |
| Ovr63A | Ovary 13 | 0.25 | | |
| Ovr35GA | Ovary 14 | | | 2.00 |
| Ovr18GA | Ovary 15 | | | 1.94 |
| Ovr206I | Ovary 16 | | | 2.11 |
| Ovr20GA | Ovary 17 | | | 1.91 |
| Ovr25GA | Ovary 18 | | | 3.78 |
| Ovr32RA | Ovary 19 | | | 1.72 |
| Ovr506B | Ovary 20 | | | 0.57 |
| OvrC007 | Ovary 21 | | | 0.28 |
| OvrC004 | Ovary 22 | | | 0.57 |
| Ovr40G | Ovary 23 | | | 0.60 |
| Ovr9RA | Ovary 24 | | | 1.82 |
| OvrC087 | Ovary 25 | | | 0.46 |
| OvrC109 | Ovary 26 | | | 2.35 |
| OvrC179 | Ovary 27 | | | 2.12 |
| Pan71XL | Pancreas | 7.14 | 1.19 | |
| Pro34B | Prostate | 19.36 | 11.84 | |
| SmIH89 | Small Intestine 1 | 13.88 | 2.20 | |
| SmI21XA | Small Intestine 2 | 1.10 | 0.50 | |
| Tst39X | Testis | 6.94 | 2.02 | |
| Utr141XO | Uterus 1 | 18.57 | 5.45 | |
| Utr23XU | Uterus 2 | 22.39 | 5.50 | |
| Utr135XO | Uterus 3 | 19.00 | 20.70 | |
| Utr85XU | Uterus 4 | 26.00 | 21.90 | |
| Bld66X | Bladder 1 | 10.74 | 1.47 | |
| End28XA | Endometrium 1 | 100.78 | 17.88 | |
| End5XA | Endometrium 2 | 14.52 | 30.80 | |
| End8XA | Endometrium 3 | 2.17 | 20.04 | |
| End65RA | Endometrium 4 | 32.67 | 7.89 | |
| End8963 | Endometrium 5 | 21.63 | 4.59 | |
| End10479 | Endometrium 6 | 130.20 | 16.10 | |
| End12XA | Endometrium 7 | 41.60 | 13.30 | |
| End3AX | Endometrium 8 | 3.00 | 7.50 | |
| End68X | Endometrium 9 | 25.00 | 35.00 | |
| End8911 | Endometrium 10 | 28.00 | 24.00 | |
| Kid80XD | Kidney 1 | 1.48 | 2.76 | |
| Kid6XD | Kidney 2 | 44.63 | 2.66 | |
| Kid5XD | Kidney 3 | 18.10 | 1.30 | |
| Kid98XD | Kidney 4 | 5.40 | 3.40 | |
| Liv15XA | Liver 1 | 0.03 | 0.08 | |
| Liv94XA | Liver 2 | 5.19 | 0.30 | |
| LngAC82 | Lung 1 | 2.23 | 2.08 | |
| LngAC88 | Lung 2 | 17.15 | 9.45 | |
| Lng60XL | Lung 3 | 35.75 | 4.99 | |
| LngAC69 | Lung 4 | 108.40 | 11.40 | |
| LngSQ32 | Lung 5 | 86.50 | 23.90 | |
| MamA06X | Mammary Gland 1 | 9.45 | 0.06 | |
| MamB011X | Mammary Gland 2 | 5.31 | 1.27 | |
| Mam47XP | Mammary Gland 3 | 0.80 | 0.20 | |
| Mam59X | Mammary Gland 4 | 23.40 | 1.40 | |
| Mam12X | Mammary Gland 5 | 5.12 | 5.43 | |
| ClnC9XR | Colon 1 | 7.52 | 2.52 | |
| ClnCM67 | Colon 2 | 1.78 | 0.30 | |
| ClnAS67 | Colon 3 | 3.71 | 12.30 | |
| ClnRC67 | Colon 4 | 9.40 | 7.80 | |
| ClnTX67 | Colon 5 | 42.40 | 3.30 | |
| StoAC44 | Stomach 1 | 12.00 | 38.00 | |
| StoAC93 | Stomach 2 | 24.00 | 62.00 | |
| StoAC99 | Stomach 3 | 62.00 | 34.00 | |
| StoMT54 | Stomach 4 | 49.00 | 19.00 | |
| StoTA73 | Stomach 5 | 99.00 | 51.00 | |

Table 1 and Table 2 represent a combined total of 132 samples in 15 different tissue types.

As shown in Table 2, all 120 samples from 14 different tissues analyzed, showed expression of Ovr107 (expression values >0.00).

Furthermore, the level of mRNA expression in cancer samples and the isogenic normal adjacent tissue from the same individual were compared, with the exception of the unmatched ovarian samples. This comparison provides an indication of specificity for cancer (e.g. higher levels of mRNA expression in the cancer sample compared to the normal adjacent). Table 2 shows overexpression of Ovr107 in 35 of 47 (74%) matching samples analyzed (ovary 1, pancreas, prostate, small intestine 1 and 2, testis, uterus 1, 2 and 4, bladder, endometrium 1, 4, 5, 6, 7, and 10, kidney 2, 3 and 4, liver 2, lung 2, 3, 4, and 5, mammary gland 1, 2, 3, and 4, colon 1, 2, 4, and 5, and stomach 3, 4, and 5).

For the unmatched ovarian samples, 11 of 13 (85%), cancer samples show expression values of Ovr107 higher than the median (1.82) for the normal unmatched ovarian samples, and 10 of 13 (77%) show expression higher than the highest level seen in normal ovary.

Altogether, the broad tissue distribution, plus the mRNA overexpression in a majority of cancer samples compared to normal are indicative of Ovr107 being a marker not only for ovarian cancer, but for cancer in general.

1. A diagnostic marker for cancer comprising Ovr107.

2. The diagnostic marker of claim 1 where Ovr107 comprises SEQ ID NO:1.

3. A method for detecting the presence of cancer in a patient comprising:
(a) determining levels of the diagnostic marker of claim 1 in cells, tissues or bodily fluids in a patient; and
(b) comparing the determined levels of the diagnostic marker of claim 1 with levels of the diagnostic marker of claim 1 in cells, tissues or bodily fluids from a normal human control, wherein a change in determined levels of the diagnostic marker of claim 1 in said patient versus normal human control is associated with the presence of cancer.

4-7. (canceled)

8. A method of identifying potential therapeutic agents for use in imaging and treating cancer comprising screening molecules for an ability to bind to the diagnostic marker of claim 1 wherein the ability of a molecule to bind to the diagnostic marker of claim 1 is indicative of the molecule being useful in imaging and treating cancer.

9. An antibody which specifically binds the diagnostic marker of claim 1.

10. A method of imaging cancer in a patient comprising administering to the patient the antibody of claim 9.

11. The method of claim 10 wherein said antibody is labeled with paramagnetic ions or a radioisotope.

12. A method of treating cancer in a patient comprising administering to the patient the antibody of claim 9.

13. The method of claim 12 wherein the antibody is conjugated to a cytotoxic agent.

14. A method of treating cancer in a patient comprising administering to the patient a molecule which downregulates expression or activity of the diagnostic marker of claim 1.

15. A method of inducing an immune response against a target cell expressing the diagnostic marker of claim 1 comprising delivering to a human patient an immunogenically stimulatory amount of an Ovr107 protein so that an immune response is mounted against the target cell.

16. A vaccine for treating cancer comprising Ovr107.


SEQUENCE LISTING (5 sequences)

SEQ ID NO: 1 - DNA, 1635 bp, Homo sapiens
  misc_feature (884)..(884): n=a, c, g or t
tcgcggccgcaggagccggcgccgggcggctggggagggcttgctgacgctgcgggccaagccgccctcggaggccgagtacaccgacgtgctgcagaagatcaagtacgccttcagcctgctggcccggctgcgcggcaacatcgccgacccctcctctccggagctgttgcacttccttttcgggcctctgcagatgattgtgaacacgtcgggggggccggagttcgcgagcagtgtgcggcggccgcatctgacatcggatgccgtggcgctgctgcgggacaacgtcactccacgtgaaaacgagctctggacctcgctgggggactcgtggacccgccccgggctggagctgtccccggaggagggacccccatacagacccgagttcttcagcggctgggagccgccggtcactgacccgcagagccgcgcctgggaggacccagttgagaaacagctacagcacgagcggaggcgccggcagcaaagcgcccccgaggtcgctgtcaatggtcaccgaggacttgggagccagaatctgagcctcagctggagtcagagacagcaggaaaatgggtcctgtgtaattatgacttccaggcccgcaacagcagtgagctgtcggtcaagcagcgggacgtactggaggtcctggatgacagtcgtaagtggtggaaggttcgggacccagcggggcaggagggatatgtgccctacaacatcctgacaccctaccccggaccccggctgcaccacagccaaagccctgcccgcagcctgaacagcactcctcctccaccaccagccccagccccggccccacctccagctctggctcggccccgctgggacaggccccgctgggacagctgcgatanctcaacggcttggaccccagcgagaaggagaaattctcccagatgctcatcgtcaacgaggaactgcaggcgcgcctggcccagggccgctcgggaccgagccgcgcagtcccagggccccgcgccccggaaccgcagctcagcccgggctcggacgcctccgangtccgcgcctggctgcaggncaagggctttagctccgggaccgtggacgcgctgggtgtgctgaccggggcgcacttttctcgctgcagaaggaggagctgcgggcggtgagccccgaggagggggcacgtgtgtacagccaggtcaccgtgcagcgcttcgctgctggaggacaaagagaaagtgtcagagctggaggcagtgatggagaagcaaaagaagaaggtggaaggcgaggtggaaatggaggtcatttgacctgccaggcgcccttcgcaaagagtgacgaggccccgtgggagaacggactcctcagactctccccaatagcggaagtcgatcttctgaaggatggccaatctgctccggccctggtcttcccccatcccggtggacagacttaacgatccttgctgcagtccctccggagaggatctggactggctgggagtggggagggcgtggagacagtctacggaaagcgctagcagacccccgagagggtgcagtggagccctgagcattgtaatatgcggcccagcctataaacagcctccgtgcttagcagatggtgtgccanttnaaa

SEQ ID NO: 2 - DNA, 21 bp, Artificial Sequence
  Synthetic
cccaatagcggaagtcgatct

SEQ ID NO: 3 - DNA, 20 bp, Artificial Sequence
  Synthetic
cactcccagccagtccagat

SEQ ID NO: 4 - DNA, 22 bp, Artificial Sequence
  Synthetic
aatctgctccggccctggtctt

SEQ ID NO: 5 - DNA, 1635 bp, Homo sapiens; identical to SEQ ID NO: 1
  misc_feature (884)..(884): n=a, c, g or t